(19) United States
(12) Patent Application Publication
(10) Pub. No.: US 2008/0161597 A1
(43) Pub. Date: Jul. 3, 2008

(54) Method For the Production of a Thioacetic Acid and Salts Thereof

(76) Inventors: Christoph Taeschler, Termen (CH); Paul Hanselmann, Brig-Glis (CH); Claudio Arnold, Brig-Glis (CH)

Correspondence Address: FISHER, CHRISTEN & SABOL, 1725 K STREET, N.W., SUITE 1108, WASHINGTON, DC, 20006, US

(21) Appl. No.: 10/546,651

(22) PCT Filed: Mar. 2, 2004
(86) PCT No.: PCT/EP2004/002089

§ 371 Date: Mar. 10, 2008

(30) Foreign Application Priority Data
Mar. 3, 2003 (EP) 03004654.4

(51) Int. Cl. C07C 327/06

(52) U.S. Cl. 562/26

(57) ABSTRACT

The invention relates to a method for the production of an thioacetic acid and salts thereof of formulae (I) and (II), wherein Mₙ₊represents ammonium or an alkali metal cation, alkaline earth metal cation, aluminum cation or titanium cation, by reacting ketene with hydrogen sulphide in the presence of a nitrogenous base or reacting ketene with an aqueous alkali metal hydrogen sulphide solution. The thioacetic acid thus formed can be, optionally, subsequently transformed into the corresponding salt by reacting it with ammonia or an alkali metal base, alkaline earth metal base, aluminum base or titanium base. The transformation of thioacetic acid and the formation of salt is carried out as a one-pot method.

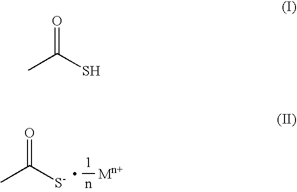

The invention relates to a process for preparing thioacetic acid and its salts.

Thioacetic acid and its salts are important industrial starting materials and are used, for example, for the preparation of D-acetylthioisobutyric acid or active pharmaceutical ingredients such as Captopril (ACE inhibitor against hypertension, JP-A-09-295963) and for the synthesis of biotin (DE-A-2807200). Potassium thioacetate is used, for example, in the preparation of testosterone derivatives (DE-A-19860917).

Known processes for the synthesis of thioacetic acid start, for example, from acetic anhydride which is reacted with hydrogen sulfide (OA-A-2110) or metal hydrogensulfides (US-A-2568020) to give the desired product.

A further process starts from ketene which is reacted with hydrogen sulfide in the gas phase and in the presence of a solid aluminum-containing catalyst at from 10 to 204.4° C. (50 to 400° F.) , preferably at from 65.6 to 176.7° C. (150 to 350° F) to give thioacetic acid or diacetyl sulfide (US-A-2639293) .

A common process for preparing alkali metal thiocarboxylates starts from thiocarboxylic acids which, for example, are converted to the corresponding salts by means of alkali metal hydrides (Kato, S. et al. , Z. Naturforsch. 1983, 38B(12), 1585-1590).

The methods disclosed to date have the disadvantage that thioacetic acid and its salts have always been prepared in separate process steps.

It is therefore an object of the present invention to provide a simple process for directly preparing thioacetic acid and its salts, which can also be conducted in a “one-pot process”, works inexpensively, utilizes readily available starting materials and affords a small amount of waste products and by-products.

According to the invention, this object is achieved by the process as claimed in patent claim 1. It has been found that ketene can be reacted in the presence of a nitrogen base directly with hydrogen sulfide or a dissolved alkali metal hydrogensulfide. Depending on the requirement, the thioacetic acid can be obtained directly in good yield and high purity by subsequent distillation, or its corresponding salt by addition of ammonia, or of an alkali metal or alkaline earth metal, aluminum or titanium base. More preferably, the formation of the thioacetic acid with subsequent salt formation can be carried out as a “one-pot process”. This allows solvent to be saved and waste substances to be prevented to a high degree.

The inventive reaction of ketene with hydrogen sulfide or a dissolved alkali metal hydrogensulfide in the absence of an aluminum catalyst is surprising, since US-A-2639293 states that, in the reaction of ketene with hydrogen sulfide in the absence of a catalyst, no detectable amounts of thioacetic acid or diacetyl sulfide are obtained. The reaction of ketene with a dissolved alkali metal hydrogensulfide has not yet been described.

In the process according to the invention for preparing thioacetic acid and its salts of the formulae

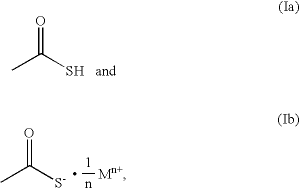

in which Mⁿ⁺is ammonium or an alkali metal, alkaline earth metal, aluminum or titanium cation, selected from the group consisting of lithium, sodium, potassium, magnesium, calcium, barium, aluminum and titanium, and in which n is the number of positive charges of the cation, ketene is reacted in the presence of a nitrogen base with hydrogen sulfide or a dissolved alkali metal hydrogensulfide, and the thus obtained thioacetic acid is optionally subsequently reacted with ammonia or an alkali metal, alkaline earth metal, aluminum or titanium base to give the corresponding salt.

Here and hereinbelow, alkali metal hydrogensulfides refer in particular to the hydrogensulfides of lithium, sodium and potassium.

Here and hereinbelow, alkali metal or alkaline earth metal, aluminum or titanium bases refer in particular to hydroxides, carbonates, hydrogencarbonates, alkoxides, phenoxides, carboxylates, oxides, hydrides, sulfonates, phosphates, sulfides, sulfinates, oxalates, hexafluorophosphates and tetrafluoroborates of lithium, sodium, potassium, magnesium, calcium, barium, aluminum and titanium.

Here and hereinbelow, alkoxides and phenoxides of alkali metals, alkaline earth metals, aluminum and titanium refer in particular to the compounds of the formula

$${R}^{1} {O}^{-} \cdot \frac{1}{n} {M}^{n +}$$

where R¹ is a C₁₋₆-alkyl or phenyl radical, and Mⁿ⁺is an alkali metal cation, preferably Li⁺, Na⁺or K⁺, an alkaline earth metal cation, preferably Mg²⁺, Ca²⁺or Ba²⁺, an aluminum cation or a titanium cation, and in which n is the number of positive charges of the cation.

Here and hereinbelow, carboxylates of alkali metals, alkaline earth metals, aluminum and titanium refer in particular to compounds of the formula

$${R}^{2} {COO}^{-} \cdot \frac{1}{n} {M}^{n +} ,$$

where R² is an optionally partly or fully halogenated C₁₋₆-alkyl or phenyl radical, and Mⁿ⁺is an alkali metal cation, preferably Li⁺, Na⁺or K⁺, an alkaline earth metal cation, preferably Mg²⁺, Ca²⁺or Ba²⁺, an aluminum cation or a titanium cation, and in which n is the number of positive charges of the cation.

Here and hereinbelow, nitrogen bases refer in particular to nitrogen compounds which have a pKₐ of >7, for example primary, secondary and tertiary alkylamines, for example di-tert-butylamine, trimethylamine and triethylamine, primary, secondary and tertiary arylamines, cyclic amines, for example piperidine, pyrrolidine or morpholine, aromatic nitrogen hetero-cycles such as pyridine or substituted pyridines, for example 4-dimethylaminopyridine, ammonia, guanidines, bicyclic nitrogen compounds, for example 1,4-diaza-bicyclo[2.2.2]octane (DABCO), 1,5-diazabicyclo-[4.3.0]non-5-ene (DBN) or 1,8-diazabicyclo-[5.4.0]undecene (DBU), and also basic nitrogen-containing ion exchangers which are obtainable, for example, under the names Lewatit®, Duolite® and Dowex®. The task of the nitrogen base is to activate the H₂S.

In a preferred embodiment, Mⁿ⁺is selected from the group consisting of Li⁺, Na⁺, K⁺, Mg²⁺, Ca²⁺and Ba²⁺.

The nitrogen base used is preferably a tertiary amine, more preferably a trialkylamine, most preferably trimethyl- or triethylamine.

In an advantageous process variant, the reaction of ketene with hydrogen sulfide or a dissolved alkali metal hydrogensulfide is carried out in a polar solvent.

In a preferred embodiment, the polar solvent used is a C₁₋₄ alcohol, water, diethyl ether, tetrahydrofuran, dichloromethane, pyridine, methylpyridine or dimethylformamide.

In a particularly preferred embodiment, the polar solvent used is a C₁₋₄ alcohol or water.

In a further particularly preferred embodiment, potassium thioacetate or sodium thioacetate is prepared by using 1-butanol as a polar solvent, in which potassium thioacetate and sodium thioacetate crystallize out particularly efficiently.

In a particular process variant, ketene can be reacted in aqueous solution with an alkali metal hydrogensulfide solution, optionally with addition of gaseous hydrogen sulfide.

In the process according to the invention, hydrogen sulfide and ketene or alkali metal hydrogensulfide and ketene are used advantageously in a molar ratio between 0.5:and 2:. In a preferred embodiment, a molar hydrogen sulfide:ketene ratio or alkali metal hydrogensulfide:ketene ratio between 0.8:and 1.3:is used. In a particularly preferred embodiment, a molar hydrogen sulfide: ketene ratio or alkali metal hydrogensulfide:ketene ratio of 1:is used.

In a preferred embodiment of the process according to the invention, a molar ketene:nitrogen base ratio between 1:0.001 and 1:0.5 is used. In a particularly preferred embodiment, a molar ketene:nitrogen base ratio between 1:0.001 and 1:0.1 is used.

In a preferred embodiment, the reaction of ketene with hydrogen sulfide is carried out at temperatures between +60 and -40° C., more preferably between +10 and -20° C.

For the formation of the salts of thioacetic acid, alkali metal, alkaline earth metal, aluminum or titanium bases may be used in the process according to the invention. The alkali metal or alkaline earth metal, aluminum or titanium bases used are preferably hydroxides, carbonates, hydrogencarbonates, alkoxides, phenoxides, carboxylates, oxides, hydrides, sulfonates, phosphates, sulfides, sulfinates, oxalates, hexafluoro-phosphates or tetrafluoroborates of lithium, sodium, potassium, magnesium, calcium, barium, aluminum or titanium. The resulting metal thioacetate may optionally be removed by filtration from the corresponding acid of the metal base used, and it is possible depending on the solubility for the metal thioacetate or the corresponding acid to be dissolved.

In a particularly preferred embodiment, a hydroxide, alkoxide, phenoxide or carboxylate of lithium, sodium or potassium is used for salt formation.

In one process variant, the alkali metal, alkaline earth metal, aluminum or titanium base may be added as a solid or as an aqueous solution and/or suspension.

In a further process variant, sparingly soluble or insoluble alkaline earth metal, aluminum or titanium bases, for example aluminum hydroxide, calcium carbonate and titanium dioxide, preferably in fine granulation, more preferably in pulverized form, may also be reacted with the thioacetic acid for salt formation.

In a further process variant, the reaction with the alkali metal or alkaline earth metal, aluminum or titanium base may be carried out at temperatures of from −20 to +100° C., more preferably at temperatures of from −10 to +10° C.

In the process according to the invention, the salt formation may also be carried out as a “one-pot process” without preceding isolation of the thioacetic acid.

### Examples

The examples which follow illustrate the performance of the process according to the invention without any implied restriction.

### Example 1

### Thioacetic Acid

A mixture of 1-butanol (1300 ml) and triethylamine (17.7 g; 0.175 mol) was initially charged and cooled to −10° C. Subsequently, hydrogen sulfide was introduced with a flow rate of 34.4 g/h in a total amount of 60.2 g (1.75 mol). After 10 min, ketene was additionally introduced with a flow rate of 42 g/h in a total amount of 99.4 g (1.75 mol). During the introduction of hydrogen sulfide and ketene, the reaction temperature was kept below −5° C. On completion of the introduction, the thioacetic acid formed was distilled off. 86 g (65%) of thioacetic acid were obtained in a purity of >98% (GC).

¹H NMR (400 MHz, DMSO) : δ=2.41 (s, 3H) ; ¹³C NMR (133 MHz, CDCl₁₃) : δ=171.6 ppm.

### Example 2

### Potassium Thioacetate

A mixture of 1-butanol (1300 ml) and triethylamine (17.7 g; 0.175 mol) was initially charged and cooled to −10° C. Subsequently, hydrogen sulfide was introduced with a flow rate of 34.4 g/h in a total amount of 60.2 g (1.75 mol). After 10 min, ketene was additionally introduced with a flow rate of 42 g/h in a total amount of 99.4 g (1.75 mol). During the introduction of hydrogen sulfide and ketene, the reaction temperature was kept below −5° C. On completion of the introduction, a 45% aqueous potassium hydroxide solution (218 g; 1.75 mol) was metered in within 20 min and the mixture was heated under reflux for 1 h. In the course of this, white potassium thioacetate precipitated out, and, after cooling of the reaction mixture to −5° C., was filtered off, washed with 1-butanol and then dried. 98.5 g (63%) of potassium thioacetate were obtained in a purity of >98% (titrimetric).

What is claimed is

1. A process for preparing thioacetic acid and its salts of the formulae: 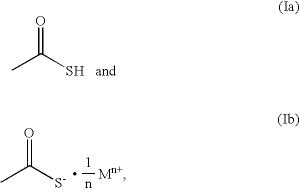
in which Mⁿ⁺is a cation selected from the group consisting of ammonium and the cations of the alkali metals, of the alkaline earth metals, of aluminum and titanium, and n is the number of positive charges of the cation, characterized in that ketene is reacted in the presence of a nitrogen base with hydrogen sulfide or a dissolved alkali metal hydrogensulfide, and the thus obtained thioacetic acid is optionally subsequently reacted with ammonia or an alkali metal, alkaline earth metal, aluminum or titanium base to give the corresponding salt.

2. The process as claimed in claim 1, characterized in that Mⁿ⁺is selected from the group consisting of Li⁺, Na⁺, K⁺, Mg²⁺, Ca²⁺and Ba²⁺.

3. The process as claimed in claim 1 or 2, characterized in that the nitrogen base used is a base from the group consisting of primary, secondary and tertiary alkylamines, primary, secondary and tertiary arylamines, ammonia, guanidines, bicyclic nitrogen heterocycles and basic ion exchangers having a pKₐ of >7.

4. The process as claimed in one of claims 1 to 3, characterized in that the nitrogen base used is a tertiary amine.

5. The process as claimed in one of claims 1 to 4, characterized in that it is performed in a polar solvent.

6. The process as claimed in one of claims 1 to 5, characterized in that the polar solvent used is a C₁₋₄ alcohol, water, dimethylformamide or an optionally substituted pyridine, preferably a C₁₋₄ alcohol or water.

7. The process as claimed in one of claims 1 to 6, characterized in that ketene is reacted in aqueous solution with an alkali metal hydrogensulfide, optionally with addition of hydrogen sulfide.

8. The process as claimed in one of claims 1 to 7, characterized in that hydrogen sulfide and ketene or alkali metal hydrogensulfide and ketene are used in a molar ratio of from 0.5:to 2:, preferably of from 0.8:1 to 1.3:1.

9. The process as claimed in one of claims 1 to 8, characterized in that ketene and nitrogen base are used in a molar ratio between 1:0.001 and 1:0.5, preferably between 1:0.001 and 1:0.1.

10. The process as claimed in one of claims 1 to 9, characterized in that the reaction of ketene with hydrogen sulfide or an alkali metal hydrogensulfide is carried out at temperatures between +60 and −40° C., preferably between +10 and −20° C.

11. The process as claimed in one of claims 1 to 10, characterized in that the alkali metal, alkaline earth metal, aluminum or titanium base used is a hydroxide, carbonate, hydrogencarbonate, alkoxide, phenoxide, carboxylate, oxide, hydride, sulfonate, phosphate, sulfide, sulfinate, oxalate, hexafluorophosphate or tetrafluoroborate of lithium, sodium, potassium, magnesium, calcium, barium, aluminum or titanium, preferably a hydroxide, alkoxide, phenoxide or carboxylate of lithium, sodium or potassium.

12. The process as claimed in one of claims 1 to 11, characterized in that the alkali metal, alkaline earth metal, aluminum or titanium base is added as a solid or as an aqueous solution and/or suspension.

13. The process as claimed in one of claims 1 to 12, characterized in that the reaction with the alkali metal, alkaline earth metal, aluminum or titanium base is carried out at a temperature of from −20to +100° C., preferably from −10 to +10° C.

14. The process as claimed in one of claims 1 to 13, characterized in that the salt formation is carried out without preceding isolation of the thioacetic acid.
